(19) United States
(12) Patent Application Publication
(10) Pub. No.: US 2004/0092431 A1
(43) Pub. Date: May 13, 2004

(54) Histone deacetylase inhibitors for treating degenerative diseases of the eye

(76) Inventor: Peggy E. Hellberg, Arlington, TX (US)

Correspondence Address: Teresa J. Schultz, Alcon Research, Ltd., 6201 South Freeway, Q-148, Fort Worth, TX, 76134-2099, US

(21) Appl. No.: 10/694,309

(22) Filed: Oct. 27, 2003

Related U.S. Application Data
(60) Provisional application No. 60/425,576, filed on Nov. 12, 2002.

(51) Int. Cl. A61K 31/00; A61K 38/00

(52) U.S. Cl. 514/2; 514/1

(57) ABSTRACT

Compositions and methods for treating degenerative conditions and diseases of the eye with histone deacetylase inhibitors are disclosed.

This application claims priority from U.S. S. No. 60/425,576, filed Nov. 12, 2002.

### BACKGROUND OF THE INVENTION

The present invention is directed to compounds which function as histone deacetylase (HDAC) inhibitors for treating persons suffering from acute or chronic degenerative conditions or diseases of the eye.

Glaucoma is a family of diseases, each of which is distinguished by a particular characteristic of that disease form. Primary open angle glaucoma (POAG) is characterized by typical glaucomatous changes to optic nerve head topography, arcurate scotomas in the visual field, an open angle, and is usually associated with elevated intraocular pressure (IOP). Normotension glaucoma (NTG) or low tension glaucoma is very similar to POAG except the IOP for these patients is in the normal range. Other forms of glaucoma include closed angle glaucoma and pigmentary dispersion glaucoma. All these forms of glaucoma are similar in that patients suffer from the continued loss of nerve fiber layer and visual field. Current therapies for the treatment of glaucoma, in particular POAG and NTG, strive to slow the progression of the visual field loss by lowering and controlling intraocular pressure. This is done either by IOP lowering drugs or by argon laser trabeculoplasty (ALT) and/or by glaucoma filtration surgery (GFS). Long-term studies of the effects of lowering IOP (even in NTG patients) have been shown to be effective in slowing the disease progression in some patients. Unfortunately, there are patients who continue to lose visual field despite having their IOP lowered.

Drug therapies that both lower IOP and provide additional protection to the retina and optic nerve head have been developed. Compounds such as betaxolol and brimonidine have been shown to be neuroprotective in animal models. Both have been suggested to provide neuroprotection in glaucoma by direct penetration to the back of the eye after topical ocular administration. Betaxolol's neuroprotection properties are believed to arise from its calcium channel blocking activities and its ability to stimulate the expression of key neuroprotective factors such as CNTF, bFGF, and BDNF. Brimonidine is an α₂ agonist and is believed to stimulate the production of bFGF.

Age-related macular degeneration (AMD) is the leading cause of blindness in the elderly, with an incidence of about 20% in adults 65 years of age increasing to 37% in individuals 75 years or older. Non-exudative AMD (Dry AMD) is characterized by drusen accumulation and atrophy of rod and cone photoreceptors in the outer retina, retinal pigment epithelium (RPE), Bruch's membrane and choriocapillaris; while exudative AMD leads to choroidal neovascularization (Green and Enger, Ophthalmol, Vol.100:1519-1535, 1993; Green et al., Ophthalmol, Vol. 92:615-627, 1985; Green and Key, Trans Am Ophthalmol Soc., Vol. 75:180-254, 1977; Bressler et al., Retina, Vol. 14:130-142, 1994; Schneider et al., Retina, Vol. 18:242-250, 1998; Green and Kuchle, In: Yannuzzi, L. A., Flower, R. W., Slakter, J. S. (Eds.), Indocyanine Green Angiography, St. Louis: Mosby, pg. 151-156, 1997). Retinitis pigmentosa (RP) represents a group of hereditary dystrophies characterized by rod degeneration with secondary atrophy of cone photoreceptors and underlying pigment epithelium. (Pruett, Trans Am Ophthalmol Soc., Vol. 81:693-735, 1983; Heckenlively, Trans Am Ophthalmol Soc., Vol. 85:438-470, 1987; Pagon, Sur Ophthalmol, Vol. 33:137-177, 1988; Berson, Invest Ophthalmol Vis Sci, Vol. 34:1659-1676, 1993; Nickells and Zack, Ophthalmic Genet, Vol. 17:145-165, 1996). The pathogenesis of retinal degenerative diseases such as AMD and RP is multifaceted and can be triggered by environmental factors in normal individuals or in those who are genetically predisposed. To date more than 100 genes have been mapped or cloned that may be associated with various outer retinal degenerations.

Light exposure is an environmental factor that has been identified as a contributing factor to the progression of retinal degenerative disorders such as AMD (Young, Sur Ophthal, Vol. 32:252-269, 1988; Taylor, et al., Arch Ophthal, Vol.110:99-104, 1992; Cruickshank, et al., Arch Ophthal, Vol. 111:514-518, 1993). Photo-oxidative stress leading to light damage to retinal cells has been shown to be a useful model for studying retinal degenerative diseases for the following reasons: damage is primarily to the photoreceptors and retinal pigment epithelium (RPE) of the outer retina, the same cells that are affected in heredodegenerative diseases (Noell et al., Invest Ophthal Vis Sci, Vol. 5:450-472, 1966; Bressler et al., Sur Ophthal, Vol. 32:375-413, 1988; Curcio et al., Invest Ophthal Vis Sci, Vol. 37:1236-1249, 1996); apoptosis is the cell death mechanism by which photoreceptor and RPE cells are lost in dry AMD and RP, as well as following a photo-oxidative induced cell injury (Ge-Zhi et al., Trans AM Ophthal Soc, Vol. 4:411-430, 1996; Abler et al., Res Commun Mol Pathol Pharmacol, Vol. 92:177-189, 1996; Nickells and Zack, Ophthalmic Genet, Vol. 17:145-165, 1996); light has been implicated as an environmental risk factor for progression of AMD and RP (Taylor et al., Arch Ophthalmol, Vol. 110:99-104, 1992; Naash et al., Invest Ophthal Vis Sci, Vol. 37:775-782, 1996); and therapeutic interventions which inhibit photo-oxidative injury have also been shown to be effective in animal models of heredodegenerative retinal disease (LaVail et al., Proc Nat Acad Sci, Vol. 89:11249-11253, 1992; Fakforovich et al., Nature, Vol. 347:83-86, 1990; Frasson et al., Nat. Med. Vol. 5:1183-1187,1990).

A number of different compound classes have been identified in various animal models that minimize retinal photo-oxidative injury. They include: antioxidants such as ascorbate (Organisciak et al., Invest Ophthal Vis Sci, Vol. 26:1589-1598, 1985), dimethylthiourea (Organisciak et al., Invest Ophthal Vis Sci, Vol. 33:1599-1609, 1992; Lam et al., Arch Ophthal, Vol. 108:1751-1752, 1990), α-tocopherol (Kozaki et al., Nippon Ganka Gakkai Zasshi, Vol. 98:948-954, 1994) and β-carotene (Rapp et al., Cur Eye Res, Vol. 15:219-232, 1995); calcium antagonists such as flunarizine (Li et al., Exp Eye Res, Vol. 56:71-78, 1993; Edward et al., Arch Ophthal, Vol. 109:554-622, 1992; Collier et al., Invest Ophthal Vis Sci, Vol. 36:S516); growth factors such as basic-fibroblast growth factor, brain derived nerve factor, ciliary neurotrophic factor, and interleukin-1-β (LaVail et al., Proc Nat Acad Sci, Vol. 89:11249-11253, 1992); glucocorticoids such as methylprednisolone (Lam et al., Graefes Arch Clin Exp Ophthal, Vol. 231:729-736, 1993) and dexamethasone (Fu et al., Exp Eye Res, Vol. 54:583-594, 1992); iron chelators such as desferrioxamine (Li et al., Cur Eye Res, Vol. 2:133-144, 1991); NMDA-antagonists such as eliprodil and MK-801 (Collier et al., Invest Ophthal Vis Sci, Vol. 40:S159, 1999).

Histone acetyltransferase/deacetylases are important players in higher order chromatin design and gene transcriptions. Acetylation of histones is associated with a transcriptionally active chromatin state; whereas, deacetylation is correlated with a closed chromatin state which would cause gene repression. It has been shown that HDAC inhibitors can reactivate gene expression and inhibit the growth and survival of tumor cells (Johnstone, Nature Reviews, Drug Discovery, Vol. 1, April 2002). HDAC inhibitors are now being tested for their usefulness as anticancer agents (e.g. FR-901228 by Fujisawa; MS-275 by Schering AG; Acetyidinaline (CI-994; PD-123654) by Pfizer; MG-2856 by MethylGene; VX-563 by Vertex). HDAC inhibitors have not been suggested for use in treating persons suffering from degenerative conditions or diseases of the eye.

### SUMMARY OF THE INVENTION

The present invention is directed to the use of HDAC inhibitors or (“Compounds”) to treat persons suffering from acute or chronic degenerative conditions or diseases of the eye, particularly: glaucoma, dry AMD; RP and other forms of heredodegenerative retinal disease; retinal detachment and tears; macular pucker; ischemia affecting the outer retina; cellular damage associated with diabetic retinopathy and retinal ischemia; damage associated with laser therapy (grid, focal, and panretinal) including photodynamic therapy (PDT); trauma; surgical (retinal translocation, subretinal surgery, or vitrectomy) or lightinduced iatrogenic retinopathy; and preservation of retinal transplants.

### DESCRIPTION OF PREFERRED EMBODIMENTS

The factors that lead to visual field loss in glaucoma are varied. There are a number of hypothesis that have been put forth over the years to explain glaucoma, however, none of these have been proven to be causative. Visual field loss is a direct consequence of the death (or dysfunction) of the neural retina, in particular retinal ganglion cells. Thus, drug therapies that protect retinal ganglion cells are considered to be useful. Given the fact that glaucoma is a poorly understood disease, it is not surprising that there are no well established animal models of the disease. Thus, models that provide insight into mechanism and drug classes that are protective of the neural retina serve as surrogate glaucoma models. The light induced retinopathy model is one of a few such models. This model helps to characterize the ability of a test item to protect the neural retina and, as such, compounds that are active in this model are said to be neuroprotective.

Acute or chronic degenerative conditions or diseases of the eye include, in addition to glaucoma, acute and chronic environmentally induced (trauma, ischemia, photo-oxidative stress) degenerative conditions of the photoreceptors and RPE cells in normal or genetically predisposed individuals. This would include, but not limited to, dry AMD, RP and other forms of heredodegenerative retinal disease, retinal detachment, tears, macular pucker, ischemia affecting the outer retina, cellular damage associated with diabetic retinopathy and retinal ischemia; damage associated with laser therapy (grid, focal and panretinal) including photodynamic therapy (PDT), thermal or cryotherapy, trauma, surgical (retinal translocation, subretinal surgery or vitrectomy) or light induced iatrogenic retinopathy and preservation of retinal transplants.

In general, for degenerative diseases, the Compounds of this invention are administered orally with daily dosage of these Compounds ranging between about 0.001 and about 500 milligrams. The preferred total daily dose ranges between about 1 and about 100 milligrams. Non-oral administration, such as, intravitreal, topical ocular, transdermal patch, subdermal, parenteral, intraocular, subconjunctival, or retrobulbar or subtenon's injection, trans scleral (including iontophoresis), or slow release biodegradable polymers or liposomes may require an adjustment of the total daily dose necessary to provide a therapeutically effective amount of the compound. The Compounds can also be delivered in ocular irrigating solutions. Concentrations should range from about 0.001 μM to about 100 μM, preferably about 0.01 μM to about 10 μM.

As stated above, the Compounds can be incorporated into various types of ophthalmic formulations for delivery to the eye (e.g., topically, intracamerally, intravitreal, or via an implant). They may be combined with ophthalmologically acceptable preservatives, surfactants, viscosity enhancers, gelling agents, penetration enhancers, buffers, sodium chloride, and water to form aqueous, sterile ophthalmic suspensions or solutions or preformed gels or gels formed in situ. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a viscosity enhancer, such as, hydroxymethylcellulose, hydroxyethylcellulose, hydroxypropylmethylcellulose, methylcellulose, polyvinyl-pyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

If dosed topically, the Compounds are preferably formulated as topical ophthalmic suspensions or solutions, with a pH of about 4 to 8. The Compounds will normally be contained in these formulations in an amount 0.001% to 5% by weight, but preferably in an amount of 0.01% to 2% by weight. Thus, for topical presentation, 1 to 2 drops of these formulations would be delivered to the surface of the eye 1 to 4 times per day according to the discretion of a skilled clinician.

Preferred HDAC inhibitors useful according to the present invention include: suberoylanilide hydroxamic acid (SAHA), MS-275, oxamflatin, trichostatin A, depsipeptides, and suberic bishydroxamate (SBHA).

The Compounds can also be used in combination with other agents for treating glaucoma, such as, but not limited to, β-blockers (e.g., timolol, betaxolol, levobetaxolol, carteolol, levobunolol, metipranolol), carbonic anhydrase inhibitors (e.g., brinzolamide, dorzolamide, acetazolamide), α₁ antagonists (e.g. nipradolol), α₂ agonists (e.g., opraclonidine and brimonidine), miotics (e.g., pilocarpine) and adrenergics (epinephrine), prostaglandin analogues (e.g., latanoprost, travoprost, unoprostone, bimatoprost, and compounds set forth in U.S. Pat. Nos. 5,889,052; 5,296,504; 5,422,368; 5,688,819; and 5,151,444, “hypotensive lipids” (e.g., compounds set forth in 5,352,708), neuroprotectants (e.g., compounds from U.S. Pat. No. 4,690,931, particularly eliprodil and R-eliprodil, as set forth in a pending application U.S. Ser. No. 06/203350, and appropriate compounds from WO94/13275, such as memantine, and serotonergics (5-HT₂ agonists), such as S-(+)-1-(2-aminopropyl)-indazole-6-ol and other 5-HT₂ agonists.

The following topical ophthalmic formulations are useful according to the present invention administered 1-4 times per day according to the discretion of a skilled clinician.

### EXAMPLE 1

| Ingr di nts | Amount (wt %) |
|---|---|
| Compound, especially SAHA | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 2

| Ingredients | Amount (wt %) |
|---|---|
| Compound, especially SAHA | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 3

| Ingr dients | Amount (wt %) |
|---|---|
| Compound, especially SAHA | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 4

| Ingredients | Amount (wt %) |
|---|---|
| Compound, especially SAHA | 0.01-2% |
| White petrolatum and mineral oil and | Ointment consistency |
| lanolin | |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

### EXAMPLE 5

10 mM IV Soluti n w/v %

| | |
|---|---|
| Compound, especially SAHA | 0.384% |
| L-Tartaric acid | 2.31% |
| Sodium hydroxide | pH 3.8 |
| Hydrochloric acid | pH 3.8 |
| Purified water | q.s. 100% |

### EXAMPLE 6

5 mg Capsules

| | mg/capsule |
|---|---|
| Ingredient | (Total Wt. mg) |
| Compound, especially SAHA | 5 |
| Lactose, anhydrous | 55.7 |
| Starch, Sodium carboxy-methyl | 8 |
| Cellulose, microcrystalline | 30 |
| Colloidal silicon dioxide | .5 |
| Magnesium stearate | .8 |

We claim:

1. A method for treating persons suffering from acute or chronic degenerative conditions or diseases of the eye which comprises administering a pharmaceutically effective amount of a histone deacetylase inhibitor.

2. The method of claim 1 wherein the condition or disease is selected from the group consisting of: glaucoma; dry AMD; RP and other forms of heredodegenerative retinal disease; retinal detachment and tears; macular pucker; ischemia affecting the outer retina; cellular damage associated with diabetic retinopathy and retinal ischemia; damage associated with laser therapy (grid, focal, and panretinal) including photodynamic therapy (PDT); trauma; surgical (retinal translocation, subretinal surgery, or vitrectomy) or light-induced iatrogenic retinopathy; and preservation of retinal transplants.

3. The method of claim 2 wherein the condition or disease is dry AMD.

4. The method of claim 2 wherein the condition or disease is glaucoma.
